(19) United States
(12) Patent Application Publication
(10) Pub. No.: US 2007/0250034 A1
(43) Pub. Date: Oct. 25, 2007

(54) Use of stellate ganglion block for the treatment of erectile dysfunction

(76) Inventor: Eugene Lipov, Chicago, IL (US)

Correspondence Address: BRIAN J. LUM;ICE MILLER LLP, SUITE 3500, 200 WEST MADISON STREET, CHICAGO, IL, 60606, US

(21) Appl. No.: 11/702,298

(22) Filed: Feb. 5, 2007

Related U.S. Application Data
(63) Continuation-in-part of application No. 11/411,362, filed on Apr. 25, 2006.

(51) Int. Cl. A61M 31/00

(52) U.S. Cl. 604/500

(57) ABSTRACT

The present invention is directed to a method for the treatment of a patient suffering from sexual dysfunction and other symptoms comprising the step of administering a stellate ganglion block to the patient to alleviate the symptoms. The stellate ganglion block may be followed by a sympathectomy to provide permanent relief.

This application is a continuation-in-part of U.S. application Ser. No. 11/411,362, filed Apr. 25, 2006, and claims priority to U.S. Provisional Application No. 60/691,055, filed Jun. 16, 2005, and U.S. Provisional Application No. 60/697,233, filed Jul. 7, 2005, herein incorporated by reference.

### BACKGROUND OF THE INVENTION

The present invention is directed to methods and kits for the treatment of hot flashes other symptoms of menopause, and other conditions including sexual dysfunction.

Hot flashes are experienced by many women undergoing menopause. A hot flash is a feeling of warmth, sometimes associated with flushing, that spreads over the body and may be accompanied by perspiration. Hot flashes may vary in severity and may last for a short period of time in some women, but can last for a decade or more in other women. Although the cause of hot flashes is not completely understood, hot flashes may be related to fluctuations of hormone levels experienced during menopause. Hot flashes also may be experienced secondary to mastectomy and other cancer-related treatments, particularly cancer treatments that affect hormone levels.

Traditionally, hot flashes have been treated with hormone therapy. Hormone replacement medications (usually estrogen or a combination of estrogen and progesterone) are effective in reducing the frequency of hot flashes and their severity. Generally, these medications decrease the frequency of hot flashes by about 80 to 90%. However, hormone replacement therapy is associated with increased risk of heart attack, stroke, blood clots, and breast cancer. Thus, alternative treatments are desirable.

A stellate ganglion block is an injection of local anesthetic into the stellate ganglion nerves—nerve tissue located in the neck on either side of the larynx. The injection of local anesthetic into these nerves blocks impulses, which may in turn reduce pain, swelling, color, and sweating changes in the upper extremity and may improve mobility. Stellate ganglion blocks are known as part of the treatment of Reflex Sympathetic Dystrophy (RSD), Sympathetic Maintained Pain, Complex Regional Pain Syndrome (CRPS), and Herpes Zoster (shingles) involving the head and face and/or upper extremities.

It has been found that the stellate ganglion block can be used to treat hot flashes. In an initial study, complete asymptomatic relief of two to five weeks has been experienced with a single stellate ganglion block, with increasingly longer periods of relief following subsequent blocks.

### SUMMARY OF THE INVENTION

The present invention is directed to a method for the treatment of a patient suffering from a menopause-related condition comprising the step of administering a stellate ganglion block to provide relief from the menopause-related condition. Illustratively, the patient is suffering from hot flashes and the stellate ganglion block alleviates the symptoms of hot flashes.

Additional features of the present invention will become apparent to those skilled in the art upon consideration of the following detailed description of preferred embodiments exemplifying the best mode of carrying out the invention as presently perceived.

### BRIEF DESCRIPTION OF THE DRAWINGS

FIG. 1 is a perspective view showing various vertebrae and nerves in the neck and needle placement for a stellate ganglion block.

### DETAILED DESCRIPTION

A ganglion is a group of nerve cell bodies. The stellate ganglion is a ganglion located in the neck, formed by the fusion of the inferior cervical and the first thoracic ganglions. These ganglions meet anterior to the C7 vertebra.

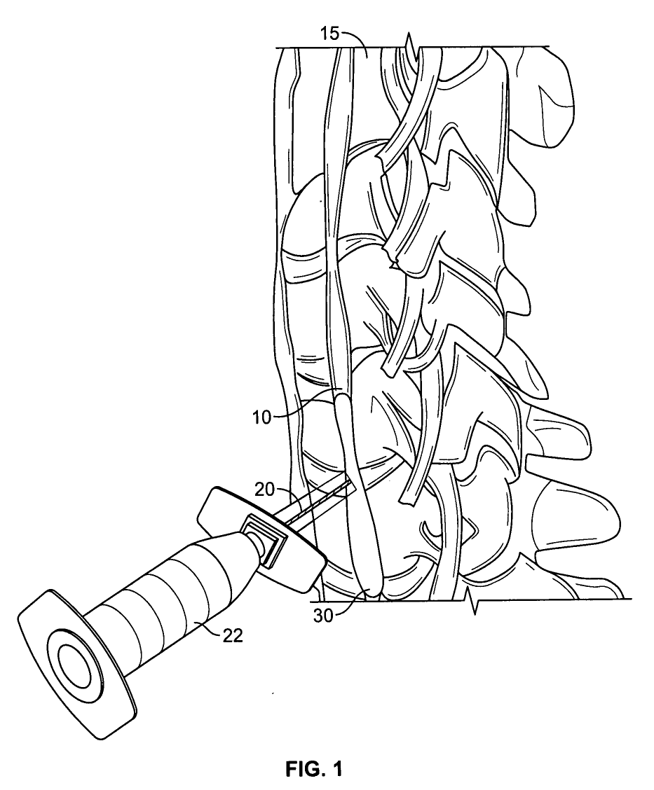

A stellate ganglion block is illustratively performed by placing the patient in the supine position (on the back) with the neck slightly extended and the jaw open. While the patient is usually conscious for the procedure, a medication such as Versed (midazolam hydrochloride), fentanyl, or other sedative is often administered, to relax the patient. A local anesthetic is also usually administered, to numb the skin and tissue down to the ganglion nerve. A needle is inserted toward the stellate ganglion. FIG. 1 shows the stellate ganglion 10 extending anterior to vertebral column 15. The needle 20 is shown inserted adjacent to the stellate ganglion 10. In the illustrative block, a contrast solution is injected through needle 20 via syringe 22, and the needle position is confirmed by fluoroscopy or other means, as are known in the art.

The anesthetic 30 is then delivered into stellate ganglion 10 by injection via needle 20. Illustratively, 5 cc 0.375% Marcaine® (bupivacaine) may be used. However, it is understood that other anesthetics, illustratively lidocaine, morphine or other opioids, clonidine, botox, or tetracine, or a combination of anesthetic and epinephrine, may be used and that the dosage range may vary depending on the particular anesthetic and other factors, as are known in the art. Needle 20 is removed, and the patient is optionally placed in the sitting position to facilitate the spread of anesthesia inferiorly to the stellate ganglion. Illustratively, longer acting anesthetics such as bupivacaine are used, but other anesthetics such as narcan may be used, as are known in the art. The stellate ganglion block may also be performed by radiofrequency lesioning of the stellate ganglion or linear polarized light irradiation near the stellate ganglion, as is known in the art.

While fluoroscopy is used in the illustrative method, stellate ganglion blocks have been traditionally performed blindly by palpating the transverse process of C6 and infiltrating a large volume (as much as 20 mL) of local anesthetic. It is understood that this technique is dependent on enough volume reaching the stellate ganglion to result in an effective block and requires a larger volume of anesthetic. While such techniques may be used to treat hot flashes, it is understood that image-guided stellate ganglion blocks have the advantages of increased safety and accuracy compared with blind injections because the needle can be accurately placed close to the ganglion itself. Because of the more accurate placement of the needle to the ganglion, a smaller amount of local anesthetic can be used, resulting in a decrease in adverse effects.

Patients may experience side effects such as nasal congestion, a hoarse voice, and a warm tingling sensation in the arm and hand, and may display symptoms associated with “Horner's syndrome” such as a bloodshot, droopy eye on the side of the injection. These side effects usually disappear after several hours. If the first injection reduces or eliminates hot flashes for a period of time, subsequent blocks may be given. It has been found that the duration of relief often increases with each subsequent injection. Surgical sympathectomy may be performed for permanent relief from hot flashes. Sympathectomies are currently performed on patients who suffer from excessive perspiration and the surgical techniques are known in the art. Generally, a sympathectomy would be performed if a patient responds well to the stellate ganglion block, to provide permanent relief from severe hot flashes.

In addition to hot flashes, it is believed that stellate ganglion blocks may aid in the treatment and/or prevention of other conditions associated with menopause, including osteoporosis, fat deposits and sexual dysfunction. For ongoing medical conditions such as osteoporosis, a series of stellate ganglion blocks may be administered periodically, for months or years. The blocks may be administered every week or two to every several months. The blocks may be administered on a regular periodic basis, or the length of time between consecutive blocks may change, illustratively increasing over time. The stellate ganglion block may also prove to be an effective treatment for overweight patients, resulting in weight loss and/or prevention or treatment of diabetes mellitus. A series of additional stellate ganglion blocks may be administered periodically, until a desired weight is attained or to maintain a desired weight.

The stellate ganglion block is also found to be useful in the treatment of sexual dysfunction, particularly those disorders experienced by women undergoing menopause or that are postmenopausal. It is well known that sexual response is controlled by the autonomic nervous system. For example, orgasm—i.e. ejaculation in men, and the smooth muscle contraction of the vagina, urethra and uterus in women—is controlled by the sympathetic branch of the autonomic nervous system, which includes the stellate ganglion. The activity of the sympathetic nervous system is modulated by hormones. For example, increased levels of estrogen are found to reduce sympathetic activity, whereas decreased levels of estrogen result in a hyperactive sympathetic system.

Menopause causes the levels of estrogen to decline appreciably and remain low for the reminder of a woman's lifetime. Without being limited to a particular theory, the reduction in estrogen levels experienced by women undergoing menopause or who are postmenopausal may create an imbalance in the activity of sympathetic nervous system that results in sexual dysfunction, such as anorgasmia—i.e. the inability to reach orgasm. The stellate ganglion block may operate by reducing the activity of the sympathetic system and restoring normal sexual response in the absence of estrogen. The repeated administration of stellate ganglion blocks over time provides extended restoration of sexual function in the same manner as the repeated treatment of hot flashes described above. Permanent relief from sexual dysfunction may be provided by surgical sympathectomy.

The stellate ganglion block is similarly effective in treatment of male erectile dysfunction or impotence. Erectile dysfunction is frequently caused by alterations in the flow of blood to the penis. In the flaccid state, the smooth muscle of the arterial and arteriolar walls in the erectile tissue are contracted under control of the sympathetic nervous system. In contrast, erection is characterized by relaxation of these smooth muscles and dilation of the arteries and arteioles in the erectile tissue under control of the parasympathetic nervous system.

Although erectile dysfunction can occur at any age, it is most common in men over 65. Impairment of the blood flow can result from a number of different physical and psychological causes, including: nerve damage, cardiovascular disorders, hormonal disorders, stress, anxiety, fatigue and other medical and psychological problems. The physical and psychological causes of erectile dysfunction are commonly distinguished by the ability to experience nocturnal or morning erections. The cause of such spontaneous erections is unclear, however, they may be related to the relaxation of smooth muscle that occurs during REM or deep sleep. The failure to experience nocturnal or morning erections typically indicates a physical rather than psychological cause of erectile dysfunction.

Erectile dysfunction is commonly treated using oral medications such as slidenafil, tadalafil or vardenafil, which enhance the effects of nitric oxide, a neurotransmitter that is believed to mediate parasympathetic vasodilation. Alternative treatments include the use of prostaglandin E, a hormone that causes relaxation of smooth muscle tissue in the penis. However, these treatments have only temporary effect and can be inconvenient to use. Furthermore, prostaglandin E must be delivered by injection or suppository, which can be painful or uncomfortable.

Similarly to the treatment of anorgasmia described above, the stellate ganglion block may operate by reducing the activity of the sympathetic system and permitting the flow of blood to the erectile tissue. The repeated administration of stellate ganglion blocks over time provides extended restoration of sexual function and permanent relief may be provided by surgical sympathectomy.

Finally, the stellate ganglion block may be effective for mood disorders, schizophrenia, and other psychiatric disorders, with repeat treatments as necessary. A sympathectomy may also be effective for these conditions. Such treatments may be particularly effective in combination with melatonin. Melatonin is a hormone produced by the pineal gland and is involved in synchronizing the body's hormone secretions and setting the brain's internal clock. Typically, the body's production of melatonin begins around dusk, increases during the night, and then decreases as daylight appears. Melatonin supplements are widely used to relieve sleep disorders, including insomnia and the symptoms of jet lag. Melatonin is also used to treat mood disorders and depression, illustratively seasonal affective disorder.

In another embodiment of the present invention, the stellate ganglion block may be used along with melatonin to treat sleep disorders, mood disorders, and depression. In one illustrative embodiment, the stellate ganglion block is performed contemporaneously with administration of melatonin. The melatonin may be administered prior to or subsequent to the stellate ganglion block, illustratively within an hour of the stellate ganglion block, but it is understood that other time periods between the administration of melatonin and stellate ganglion block are within the scope of this invention. Melatonin is often administered orally, but other dosage forms are known and may be used within the scope of this invention. Illustratively, this treatment is performed in the morning, when melatonin levels are lowest. However, this treatment may be effective at other times of the day.

### EXAMPLE 1

Six women suffering from severe menopausal hot flashes each received a standard stellate ganglion block (0.375% Marcaine, 5 cc), as described above. Hot flashes were assessed by self-reporting pre and post stellate ganglion block.

The initial stellate ganglion block was successful in all six subjects, as evidenced by a positive Horner's syndrome and anhydrosis. Successful stellate ganglion block resulted in complete alleviation of hot flashes for times ranging from 2-5 weeks. Patients returned for a follow-up stellate ganglion block after mild hot flashes returned. A second stellate ganglion block produced additional asymptomatic periods of relief ranging from 4-18 weeks. In each case, repeated blocks provided hot flash relief equal to or greater than that of the initial block. Two patients who submitted for a third stellate ganglion block reported 15 and 48 weeks relief.

Accordingly, stellate ganglion blocks appear to be related to relief of hot flashes. Repeat stellate ganglion block results in efficacious multiple week relief of severe hot flashes associated with menopause.

### EXAMPLE 2

One subject treated in accordance with Example 1, was a 42 year old woman who further suffered from sexual dysfunction, including anorgasmia. Following the administration of a stellate ganglion block, the subject experienced the return of normal sexual function. The subject continued to receive additional stellate ganglion blocks over the course of 3 years and was found to maintain normal sexual function in the same manner as experienced for treatment of hot flashes. Variations in treatment—i.e. administration of the stellate ganglion block through C7 or the use of epinephrine in addition to bupivacaine—were found to be ineffective, suggesting that the target for alleviating sexual dysfunction may be more specific than the target for treatment of hot flashes.

### EXAMPLE 3

A 36 year old man suffering from a history of erectile dysfunction for approximately 1½ years, including the absence of nocturnal and morning erections, was treated by C-6 stellate ganglion block as described above. Following the administration of a stellate ganglion block, the return of normal sexual function was essentially immediate with the subject experiencing morning erections the next day.

Although the invention has been described in detail with reference to preferred embodiments, variations and modifications exist within the scope and spirit of the invention as described and defined in the following claims.

1. A method for treating a patient experiencing erectile dysfunction, comprising the step of:
administering a stellate ganglion block to the patient to provide relief from the erectile dysfunction.

2. The method of claim 1, wherein the stellate ganglion block is administered by delivering an anesthetic to the patient's stellate ganglion.

3. The method of claim 2, wherein the anesthetic is selected from the group consisting of bupivacaine, narcan and combinations thereof.

4. The method of claim 1, wherein the stellate ganglion block is administered by surgical sympathectomy of the patient's stellate ganglion.

5. The method of claim 1, wherein the stellate ganglion block is administered by radiofrequency lesioning of the patient's stellate ganglion.

6. The method of claim 1, wherein the stellate ganglion block is administered by linear polarized light irradiation near the patient's stellate ganglion.

7. A method for treating erectile dysfunction in a patient, comprising the steps of:
administering a plurality of stellate ganglion blocks to the patient over time to provide relief from erectile dysfunction, the stellate ganglion blocks administered by delivery of an anesthetic to the stellate ganglion.

8. The method of claim 7, wherein the stellate ganglion blocks are administered on a regular periodic basis.

9. The method of claim 7, wherein the length of time between consecutive stellate ganglion blocks is increasing over time.

10. The method of claim 7, wherein the length of time between consecutive stellate ganglion blocks is at least two weeks.

11. The method of claim 7, wherein the relief from erectile dysfunction is temporary and the stellate ganglion blocks are administered after each recurrence of erectile dysfunction.

12. The method of claim 7, wherein the anesthetic is selected from the group consisting of bupivacaine, narcan and combinations thereof.

13. The method of claim 7, wherein an initial stellate ganglion block is administered by delivery of an anesthetic to the patient's stellate ganglion and a subsequent stellate ganglion block is administered by a method selected from the group consisting of: sympathectomy or radiofrequency lesioning of the patient's stellate ganglion, or linear polarized light irradiation near the patient's stellate ganglion.
